(19) United States
(12) Patent Application Publication
(10) Pub. No.: US 2022/0142476 A1
(43) Pub. Date: May 12, 2022

(54) AN APPARATUS FOR ACQUIRING SIGNALS FROM A PREGNANT SUBJECT

(71) Applicant: Biorithm Pte. Ltd., Singapore (SG)

(72) Inventors: Amrish NAIR, Singapore (SG); Diane Davis ANSERMET, Singapore (SG); Vilasini NAIR, Singapore (SG); Sihem TEDJAR, Singapore (SG)

(73) Assignee: Biorithm Pte. Ltd., Singapore (SG)

(21) Appl. No.: 17/431,057

(22) PCT Filed: Feb. 14, 2020
(86) PCT No.: PCT/SG2020/050075

§ 371 Date: Aug. 13, 2021

(30) Foreign Application Priority Data
Feb. 15, 2019 (SG) 10201901341V

(51) Int. Cl. A61B 5/00; A61B 5/276; A61B 5/282; A61B 5/344; A61B 5/391

(52) U.S. Cl. CPC A61B 5/0011; A61B 5/303; A61B 5/282; A61B 5/344; A61B 5/391; A61B 5/4356; A61B 5/6823; A61B 5/6842; A61B 5/746; A61B 2560/0412; A61B 2560/045; A61B 2562/043; A61B 2562/066; A61B 2562/164; A61B 2562/166; A61B 2562/222; A61B 2562/227; A61B 5/276

(57) ABSTRACT

An apparatus for acquiring signals from a pregnant subject includes a flexible portion that is conformable to an abdomen of the subject. The flexible portion includes a plurality of signal acquisition points, each signal acquisition point comprising a contact adapted to electrically couple to a respective electrode and signal acquisition circuitry. The flexible portion includes a first linear section that is generally aligned along a first axis; and a second linear section extending from the first linear section, the second linear section being generally aligned along a second axis that is perpendicular to the first axis; wherein a first group of the signal acquisition points is located along the first axis and a second group of the signal acquisition points is located along the second axis.

### TECHNICAL FIELD

The present invention relates to an apparatus for acquiring signals, such as electrocardiogram (ECG) or electrohysterogram (EHG) signals, from a pregnant subject.

### BACKGROUND

Prenatal care is important throughout the course of pregnancy in order to minimize the risk of complications. This typically includes regular check-ups with a healthcare professional of which prenatal examinations are usually carried out. One of the parameters that is typically measured during these check-ups is fetal heart rate. Before labour, this is typically done by means of cardiotocogram (CTG), which consists of a cardio and toco transducer. The cardio transducer, which utilises Doppler ultrasound technology, is used to monitor the fetal heart rate pattern as a function of time, allowing monitoring of the fetal wellbeing and the evaluation of any signs of fetal distress. The toco transducer, utilizing pressure sensors, measures the mechanical force exerted by the uterine muscle allowing for observation of patterns of uterine activity. This can also be done relatively cheaply with a stethoscope.

However, Doppler ultrasound devices are typically expensive and are preferably used by a trained health care professional to ensure correct operation. The Food & Drug Administration in the United States of America issued a formal statement recommending against at-home use of fetal doppler systems due to the risks of system misuse which could result in misinterpretation of fetal heart rate, over-heating fetal tissue and introducing stress on the fetus.

Another method, using electrophysiological signals such as electrocardiogram (ECG) and electrohysterogram (EHG), has the potential to address the above issues. The recording of abdominal fetal electrocardiogram (AFECG) signals is a simple and non-invasive method for monitoring the electrical activity of the fetus heart. Like standard ECG, which reflects cardiac and metabolic activity, FECG is a potentially more sensitive indicator of fetal health state compared to Doppler ultrasound given the higher resolution of heart rate and heart rate variability data that can be obtained.

Such recordings of AFECGs are typically complicated by the existence of noise, which deteriorates the signal's quality, thereby decreasing the signal-to-noise ratio (SNR). The sources of interference when acquiring the abdominal signals include: the maternal ECG, myographic noise, powerline interference, fluctuation of the baseline, and factors related to the gestation week. Furthermore, the fetal signal is rather small due to the size of the fetal heart and the intervening tissue.

It is possible to hear fetal heartbeat by means of a fetal stethoscope, sometimes referred to as a pinard horn. However, this is cumbersome because to obtain the fetal heart rate, the user would have to count the number of heart beats per minute. This may often result in inaccurate readings, and users who are unfamiliar with operating a stethoscope may not be able to locate the appropriate placement of the stethoscope to obtain an accurate and consistent reading. Additionally, the stethoscope only provides a snapshot of the fetal heart rate, and is unable to provide critical information about fetal distress.

In addition, uterine activity is typically monitored using pressure sensors positioned over the abdomen. Pressure sensing methods include the use of tocodynamometers (toco) or simple manual palpation. Tocodynamometers measure the frequency and duration of uterine contractions but have limited sensitivity when measuring the intensity of the contractions. Typically, these devices need to be sufficiently secured on the subject's abdomen to ensure minimum sensitivity to external disturbances, which usually requires a medical professional to ensure accurate positioning.

Therefore, most parents and healthcare professionals only have access to fetal heart rate and uterine contraction data during routine check-ups. The frequency of the check-ups may not be regular enough to pick up irregularities with the pregnancy which may only present intermittently, and therefore not necessarily during the check-up. Any signs of risk during the pregnancy may increase the need for more frequent check-ups at a hospital, for example, and if the check-up is inconclusive, may require hospitalization for further observation.

In certain remote locations, access to healthcare providers may be problematic and as such, pregnant women may not have access to regular check-ups. This may be due to the distance to the nearest hospital being too far or a lack of funds to afford a pre-natal check-up.

It is generally desirable to overcome or ameliorate one or more of the above described difficulties, or to at least provide a useful alternative.

### SUMMARY OF THE INVENTION

In accordance with the present invention, there is provided an apparatus for acquiring signals from a pregnant subject, the apparatus including a flexible portion that is conformable to an abdomen of the subject, the flexible portion including a plurality of signal acquisition points, each signal acquisition point comprising a contact adapted to electrically couple to a respective electrode and signal acquisition circuitry, wherein the flexible portion includes:
a first generally linear section that is generally aligned along a first axis; and a second generally linear section extending from the first linear section, the second linear section being generally aligned along a second axis that is perpendicular to the first axis;
wherein a first group of the signal acquisition points is located along the first axis and a second group of the signal acquisition points is located along the second axis.

An advantage of locating the signal acquisition points along two orthogonal axes (in substantially T-shaped fashion) is that the contact surface area between the apparatus and the skin of the subject during AFECG data acquisition is minimised without significantly compromising signal quality, when compared with previously used arcuate arrangements of signal acquisition points.

The apparatus may have a channel defined therethrough for aligning the apparatus with a navel of the subject. Advantageously, this enables the apparatus to be correctly positioned on the subject with the signal acquisition points aligned to optimise signal acquisition.

The channel may be positioned along the second axis, and may define a longitudinal axis that is mutually orthogonal to the first axis and the second axis.

At least one of the second group of signal acquisition points may be located on one side of the channel, and at least one of the second group of signal acquisition points may be located on the other side of the channel, such that signals may be acquired both from above the navel (for example, to monitor maternal and fetal ECG signals), and below the navel. For example, one of the signal acquisition points on the other side of the channel may be a uterine activity signal acquisition point, and in some embodiments, the uterine activity signal acquisition point is spaced about 8 cm from the intersection between the first axis and the second axis.

Additionally, or alternatively, one of the signal acquisition points on the other side of the channel may be a reference signal acquisition point located along the second axis to acquire reference signals, and in some embodiments, the reference signal acquisition point is located about 18 cm from the intersection between the first axis and the second axis.

Advantageously, the channel may be shaped to allow a user to align the channel to the navel of the subject by visual and/or tactile means. For example, the channel may have a chamfered entry portion to facilitate gripping by a user, to more easily enable the apparatus to be manipulated into position for application to the user.

The first group of signal acquisition points may include three signal acquisition points located along the first axis and spaced from each other by about 6 cm.

The apparatus may include a cable hub attached to the flexible portion for feeding signals from the signal acquisition points to signal acquisition circuitry and/or signal processing circuitry. The cable hub may formed from a rigid material, for example.

In embodiments that include a channel, the channel may extend through the cable hub and the flexible portion.

Some embodiments include a signal processing module attached to or integral with the cable hub, the signal processing module comprising signal processing circuitry.

In some embodiments, the apparatus comprises circuitry that is configured to determine a signal quality index (SQI) for each of a plurality of signals acquired at the signal acquisition points; and to generate an alert for any signal acquisition points for which the SQI falls below a predetermined threshold. This may be advantageous when, for example, a user is attempting to attach the apparatus to themselves for self-monitoring, but electrodes of the apparatus are not contacting the skin properly such that good quality signals cannot be obtained. The user may, in such circumstances, be alerted so that they can reposition or reattach the electrodes accordingly.

In another aspect, there is provided a system for acquiring signals from a pregnant subject, comprising:
a plurality of electrodes disposed on a flexible portion that is conformable to an abdomen of the subject, the plurality of electrodes being arranged in a T-shaped pattern in which a first group of electrodes is aligned along a first axis and a second group of electrodes is aligned along a second axis that is perpendicular to the first axis;
a rigid body that is connected to the flexible portion for alignment of the flexible portion on the abdomen of the subject; and
signal acquisition circuitry electrically coupled to said electrodes for acquiring electrocardiographic and/or electromyographic signals from the subject.

In a further aspect, there is provided a system for acquiring signals from a pregnant subject, comprising an apparatus as disclosed herein, and a plurality of electrodes, respective electrodes being attached to respective contacts of the apparatus.

### BRIEF DESCRIPTION OF THE DRAWINGS

Certain embodiments of the invention are hereafter described, by way of non-limiting example only, with reference to the accompanying drawings in which:

FIG. 1a is a back (user-facing) view of an apparatus for acquiring signals from a pregnant subject;

FIG. 1b is a front view of the apparatus;

FIG. 2a is a back perspective view of the apparatus;

FIG. 2b is a front perspective view of the apparatus;

FIG. 3 is a section view through the line A-A of FIG. 2b;

FIG. 4 is a perspective view of a signal processing module of the apparatus;

FIG. 5 is a partial exploded view of the apparatus;

FIG. 6a is a perspective view of the signal processing module being coupled to the remainder of the apparatus;

FIG. 6b is a further perspective view of the signal processing module being coupled;

FIG. 7 is a front perspective view of the apparatus, depicting relative positioning of electrodes that are to be attached to the apparatus;

FIG. 8a is a schematic view of an initial pattern used to assess electrode placement of the apparatus;

FIG. 8b is a schematic view of an example placement of electrodes;

FIG. 9 is a schematic view of the example placement of electrodes;

FIG. 10a is a perspective view of the apparatus being placed on the belly of a pregnant subject;

FIG. 10b is a perspective view of the apparatus in place on the belly of the pregnant subject;

FIG. 11a shows a back (patient-facing) view of another embodiment of an apparatus for acquiring signals from a pregnant subject;

FIG. 11b shows a front perspective view of the apparatus of FIG. 11a; and

FIG. 12 shows a block architecture of a signal processing module of an embodiment of an apparatus for acquiring signals from a pregnant subject.

### DETAILED DESCRIPTION

Embodiments of the present invention relate to an apparatus for non-invasive monitoring of maternal and fetal health by acquiring signals from a pregnant subject. Advantageously, embodiments of the apparatus provide greater ease of use for healthcare professionals in acquiring AFECG data, and may even be used for self-monitoring during pregnancy.

In at least some embodiments, apparatus 10 is wireless (in that it can acquire and process signals without a wired connection to an external computer system or power source) and/or is a wearable device.

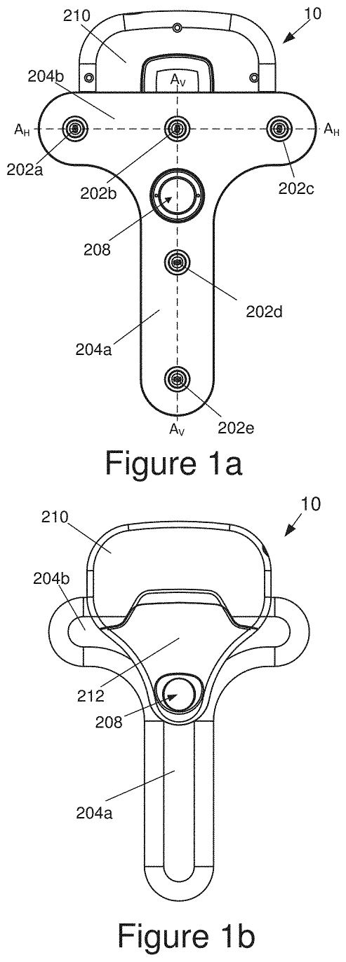

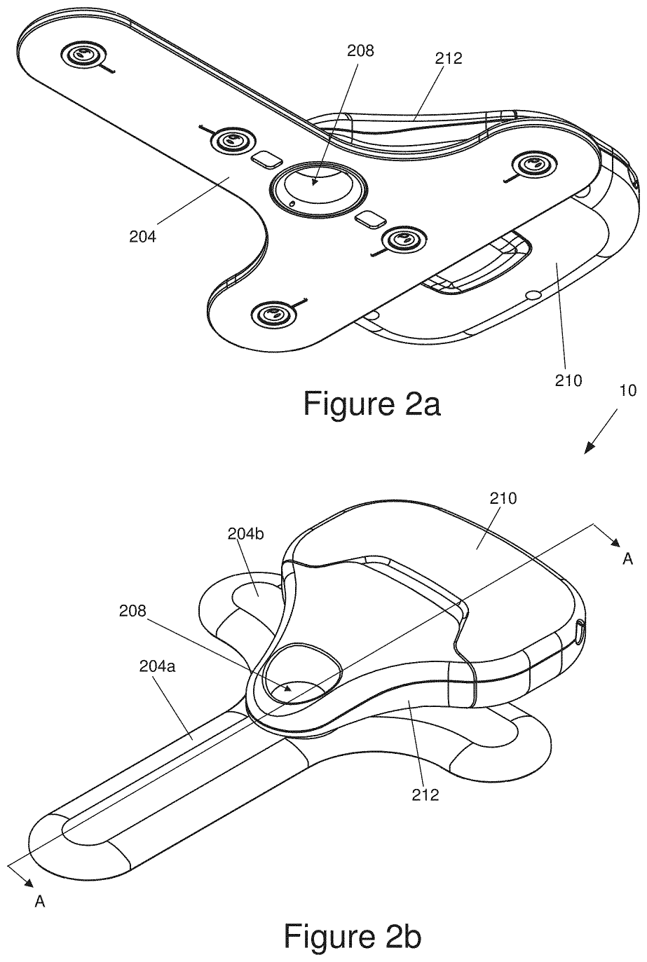

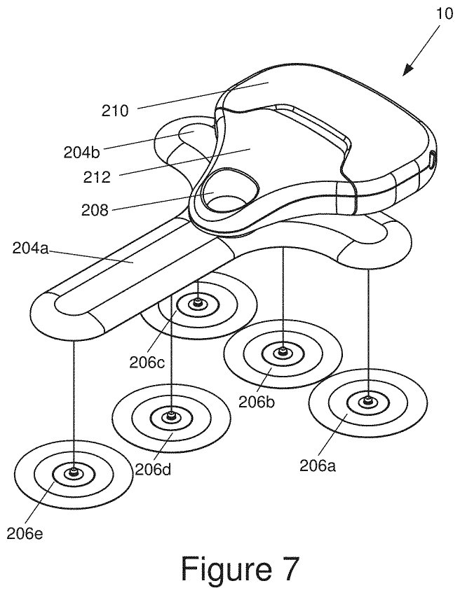

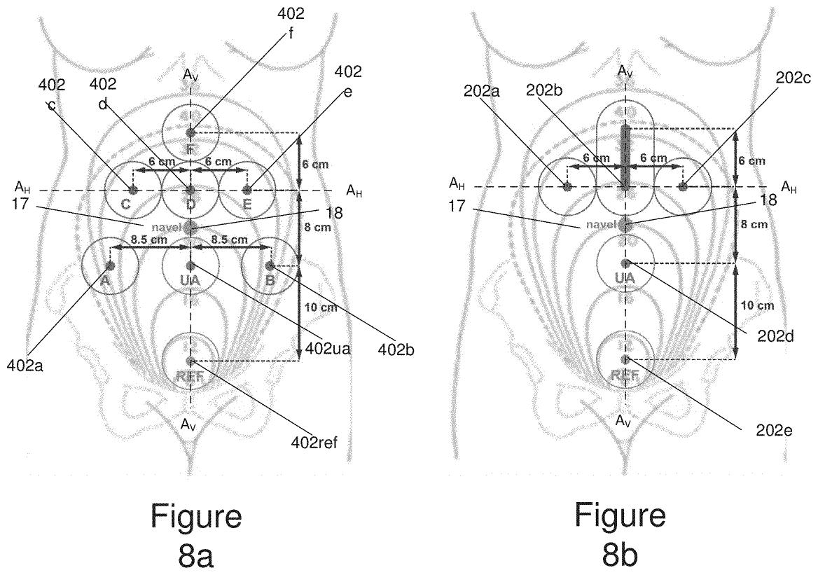

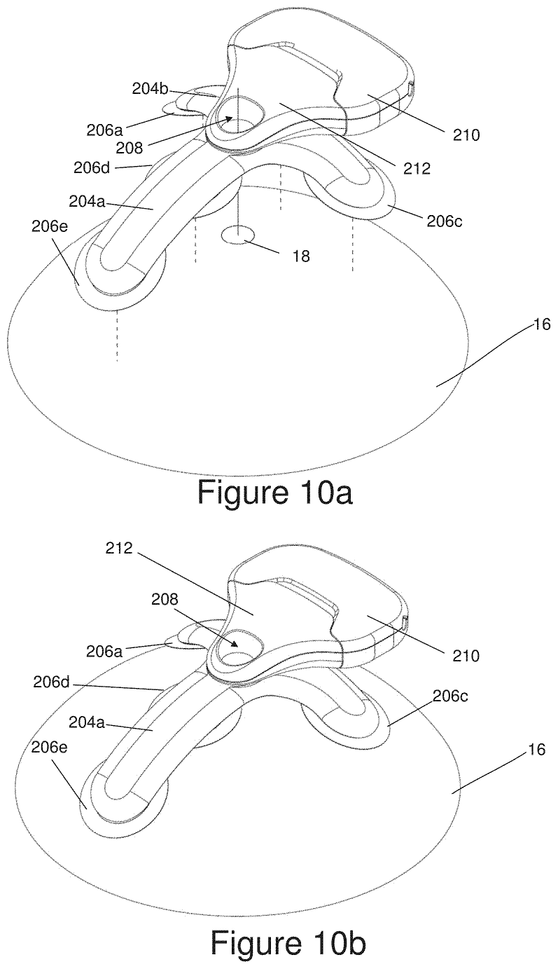

Referring initially to FIGS. 1a and 1b, an embodiment of an apparatus 10 for acquiring signals from a pregnant subject includes a flexible portion 204 (also referred to herein Referring initially to FIGS. 1a and 1b, an embodiment of an apparatus 10 for acquiring signals from a pregnant subject includes a flexible portion 204 (also referred to herein as a spreader) that is conformable to an abdominal surface of the subject (the subject 16 being depicted in FIG. 10b wearing the apparatus 10). The flexible portion 204 includes a plurality of signal acquisition points 202, each signal acquisition point 202 comprising a contact adapted to electrically couple to a respective electrode 206. For example, with reference to FIGS. 2a and 7, signal acquisition point 202a couples to electrode 206a, signal acquisition point 202b couples to electrode 206b, and so on. Each signal acquisition point 202 is also coupled to signal acquisition circuitry via one or more electrical leads. The signal acquisition circuitry may be located within the flexible portion 204 itself, and/or within a hub 212 of the apparatus 10, and/or within a signal processing module 210 of the apparatus 10.

The flexible portion 204 includes a first generally linear section 204b that is generally aligned along a first axis A_{H}; and a second generally linear section 204a extending from the first linear section 204b. The second linear section 204a is generally aligned along a second axis A_{V} that is perpendicular to the first axis A_{H}. A first group 202a, 202b, 202c of the signal acquisition points 202 is located along the first axis A_{H} and a second group 202b, 202d, 202e of the signal acquisition points is located along the second axis A_{V}.

The apparatus 10 makes monitoring maternal and fetal health status simpler, faster and more convenient. Maternal and fetal health can be monitored by parameters such as maternal heart rate, fetal heart rate and uterine activity.

For example, the signal acquisition points 202 may be integrated into the flexible portion 204 to ensure the correct positioning of the apparatus 10 when placed on the abdomen of the subject 16. In some embodiments, the signal acquisition points 202 are the female side of standard ECG snap buttons, and connect to the male connectors of electrodes 206 using a press-push (snap fit) mechanism. The signal acquired by the electrodes 206 includes but is not limited to ECG and EHG signals from unipolar leads and measurement of the signal may be relative to a common reference for multiple leads. For example, reference signal acquisition point 202e may act as a common reference for measuring signals from four unipolar leads (potential differences between respective signal acquisition points 202a, 202b, 202c, 202d and the common reference signal acquisition point 202e). It has been found by the present inventors that in the presently described embodiments, unipolar leads provide better signal quality than bipolar leads (e.g., signals measured between acquisition points 202a and 202b). The unipolar lead measures a volumetric signal, meaning the fetal heart electrical rhythm propagated would be captured at a single point as an electric field propagated through a volumetric space, and this is important given the fact that the fetus moves and the heart will be positioned at various locations within the uterus. Bipolar leads measure particular vectors as specified as the potential difference between two electrodes and this may reduce the chances of picking up fetal cardiac electric activity given the movement of the fetus.

For example, the signal acquisition points of the first group may be located symmetrically about the second axis A_{V} along the first axis A_{H}. As shown in FIG. 1a, flexible portion 204 includes 5 signal acquisition points 202, with 3 signal acquisition points 202a, 202b, 202c being arranged along the first axis A_{H} and 2 further signal acquisition points 202d, 202e being arranged along the second axis A_{V}. In this embodiment, one of the 5 signal acquisition points is a central signal acquisition point 202b and is located at the intersection of the first axis A_{H} and the second axis A_{V}.

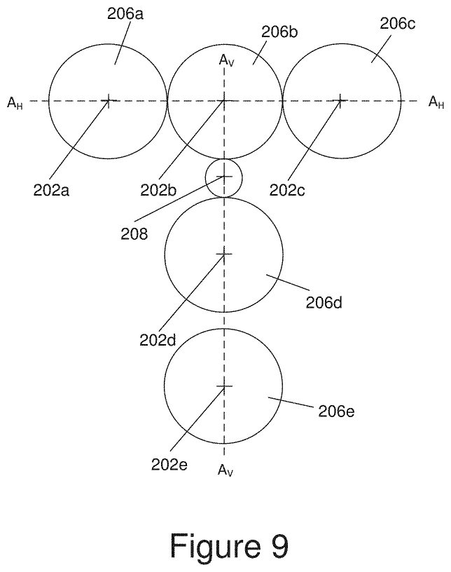

FIG. 9 shows a configuration of signal acquisition points 202 along the second axis A_{V} and first axis A_{H}. The leader lines from reference numerals 202a, 202b, 202c, 202d, 202e in FIG. 9 point to the respective centres of the signal acquisition points, while reference numerals 206a, 206b, 206c, 206d and 206e indicate the footprint of the respective electrodes to which the signal acquisition points are coupled.

In some embodiments, more than 5 signal acquisition points 202 may be arranged on the flexible portion 204. In other embodiments, fewer than 5 signal acquisition points 202 may be arranged along the axes of flexible portion 204.

As shown in FIG. 7, electrodes 206a, 206b, 206c, 206d and 206e are positioned and respectively coupled to signal acquisition points 202a, 202b, 202c, 202d and 202e in a T-shaped configuration. In some embodiments, an electrode 206 may be a disposable wet adhesive electrode such as electrodes developed by 3M™ and made available under the trade mark Red Dot™ 2238 or 2239.

In some embodiments, the first group includes three signal acquisition points 202a, 202b, 202c located along the first axis and spaced from each other by about 6 cm as shown in FIG. 8a. Advantageously, the one or more signal acquisition points are configured, when coupled to respective electrodes and signal acquisition circuitry, to acquire unipolar signals, using a common reference such as reference acquisition point 202e.

In some embodiments, a uterine activity signal acquisition point 202d is positioned along the second axis A_{V} to acquire signals associated with the pregnant subject's uterine activity. As shown in FIG. 8b, the uterine activity signal acquisition point 202d may be spaced about 8 cm from the intersection between the first axis A_{H} and the second axis A_{V}.

As shown in FIG. 8b, a reference signal acquisition point 202e is located along the second axis A_{V} to acquire reference signals. For example, the reference signal acquisition point 202e may be located about 18 cm from the intersection between the first axis A_{H} and the second axis A_{V}.

In some embodiments, the apparatus 10, and in particular flexible portion 204, has a channel 208 defined therethrough for aligning the apparatus 10 along the abdomen of the subject 16. The channel 208 acts as a tactile alignment feature, since even a non-expert user can insert their finger into the channel 208, and feel the position of the subject's navel to correctly and easily position the apparatus 10 on the abdomen in a manner which minimizes noise within the signals acquired by the electrodes 206. Advantageously, the channel 208 is shaped to allow a user to align the channel 208 to a navel 18 of the pregnant subject 16 by visual and/or tactile means, as shown in FIGS. 10a and 10b.

Advantageously, the channel 208 is positioned along the second axis A_{V}. As shown in FIG. 9, the channel 208 is positioned between the uterine activity signal acquisition point 202d and the intersection between the first axis A_{H} and the second axis A_{V}. This means that the channel 208 may be readily aligned over the navel of the subject 16 to ensure that signals are acquired both above (by signal acquisition point 202b) and below (by signal acquisition point 206d) the navel. In some embodiments, the channel defines a longitudinal axis that is mutually orthogonal to the first axis A_{H} and the second axis A_{V}.

As shown throughout the Figures a cable hub 212 is attached to the flexible portion 204 for feeding signals from the signal acquisition points to signal acquisition circuitry and/or signal processing circuitry. Cable hub 212 is electrically and mechanically coupled to second section 204b of flexible portion 204 by means of a distributor 213 (FIG. 5), for example. Cable hub 212 is further mechanically coupled to the flexible portion 205 by a ring-shaped connector 205. For example, the connector 205 may be threaded, and may mate with corresponding threads on the internal surface of the channel of cable hub 212. Alternatively, connector 205 may be arranged to form a one-way push-fit connection with the cable hub 212 to substantially permanently connect the flexible portion 204 to the cable hub 212.

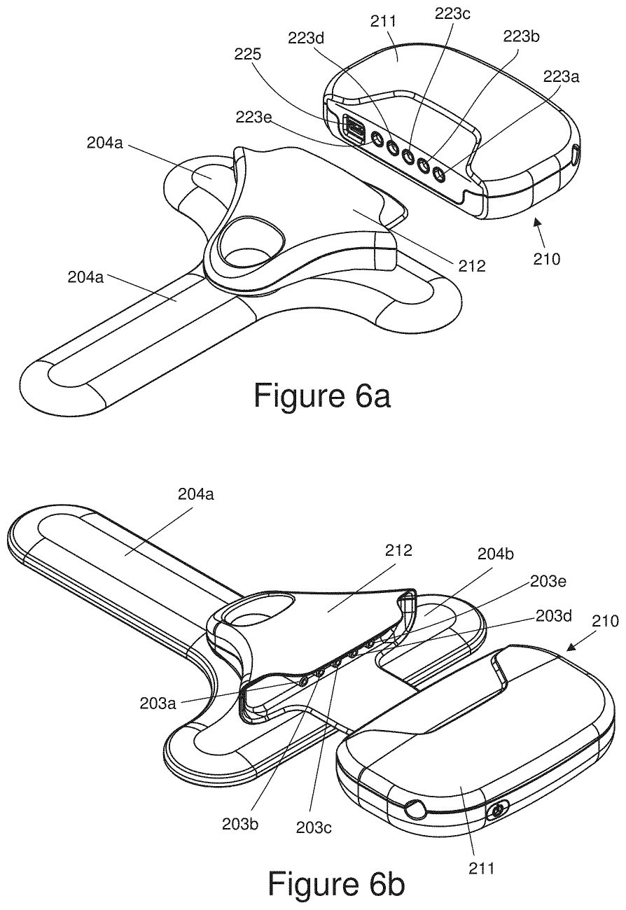

Cables or other conductors (not shown) may be embedded in flexible portion 204 and extend from respective signal acquisition points 202 to termination points of the distributor 213, which in turn are in electrical communication with respective output terminals 203a, 203b, 203c, 203d, 203e (collectively referred to hereafter as connectors 203) of the cable hub 212 (FIG. 6b).

Advantageously, cable hub 212 is formed from a rigid material, for example ABS or any other hard polymer, and forms a single body with the flexible portion 204 for providing structural integrity to apparatus 10. The cable hub 212 houses connectors 203 which, as mentioned above, are electrically coupled to the signal acquisition points 202 (for example, by wires or other conductors that extend from the signal acquisition points 202, within the flexible portion 204, terminating at connectors 203). Cable hub 212 is the male component of the connector providing electrical connection to the electrodes 206.

The cable hub has a channel extending therethrough, and being aligned with the channel 208 of the flexible portion 204 (the channel passing through both cable hub 212 and flexible portion 204 is referred to collectively as channel 208 herein). In some embodiments, the channel of the cable hub 212 has a chamfered entry portion 209 for receiving a finger of a user (such as the subject 16 themselves, for self-alignment and attachment of the apparatus 10). The chamfered entry portion 209 makes it easier for a user to insert their finger into the channel 208 for tactile alignment with the subject's 16 navel, and moreover, allows the apparatus 10 to be more easily maneuvered in that the user's finger is disposed at an angle rather than being constrained to be vertical. Additionally, the rigidity of cable hub 212 assists the user to grip the apparatus 10, in the region of the chamfered portion 209 for example, to manipulate the apparatus 10 into the correct position.

In some embodiments, the apparatus 10 includes a signal processing module 210 attached to the cable hub 212, as shown throughout the Figures. As shown in FIGS. 6a and 6b, the signal processing module 210 is coupled to the flexible portion 204, for example via cable hub 212. In some embodiments, the signal processing module 210 is detachable from the cable hub 212. The signal processing module 210 includes a housing 211 within which is housed the signal processing circuitry and preferably also at least part of the signal acquisition circuitry. Advantageously, cable hub 212 allows a secure coupling between section 204b and signal processing module 210 to maintain the stability of the device and rigidity of the connections. The housing 211 contains at least one printed circuit board (PCB) (not shown) carrying the signal processing circuitry and/or the signal acquisition circuitry, and one or more cables in communication with the one or more respective signal acquisition points 202, via ports 223a, 223b, 223c, 223d and 223e which couple to respective connectors 203a, 203b, 203c, 203d, 203d of the cable hub 212. The signal processing module 210 is preferably rigid, for example made of a material such as ABS or any other hard polymer, to store at least some of the electronic components for apparatus 10.

The first linear section 204b and second linear section 204a of the flexible portion 204 are made of a flexible material that is deformable for conforming to the abdomen 17 of the subject 16. For example, the flexible material is a flexible polymer such as a thermoplastic material (e.g. thermoplastic elastomer, TPE, or thermoplastic urethane, TPU) or silicone rubber.

Advantageously, the one or more electrodes acquire electrocardiogram (ECG) and electrohysterogram (EHG) signals from the skin of the abdomen of the subject 16. In some embodiments, the signal acquisition circuitry includes an analog front end which contains a biosignal amplifier, unipolar input for 8 channels with a minimum acquisition of 1.5 mV, for example. Further, an analog-digital convertor (ADC) and processor may be provided for converting and processing the signals received by the electrodes 206. The ADC and processor may have the following specifications: minimum resolution of 24-bits and 1 μA, (ECG), 6 μA (EHG); minimum sampling rate of 250 Hz (ECG), 100 Hz (EHG); ideal sampling rate=1000 Hz (ECG), 1000 Hz (EHG).

The signals acquired by the one or more electrodes 206 may be sent wirelessly to a transceiver of a mobile computing device (not shown) running a software application that displays, and may optionally perform post-processing on, the acquired signals. For example, wireless communication may be by Bluetooth™. That is, apparatus 10 may include a Bluetooth transmitter for communicating with the mobile computing device.

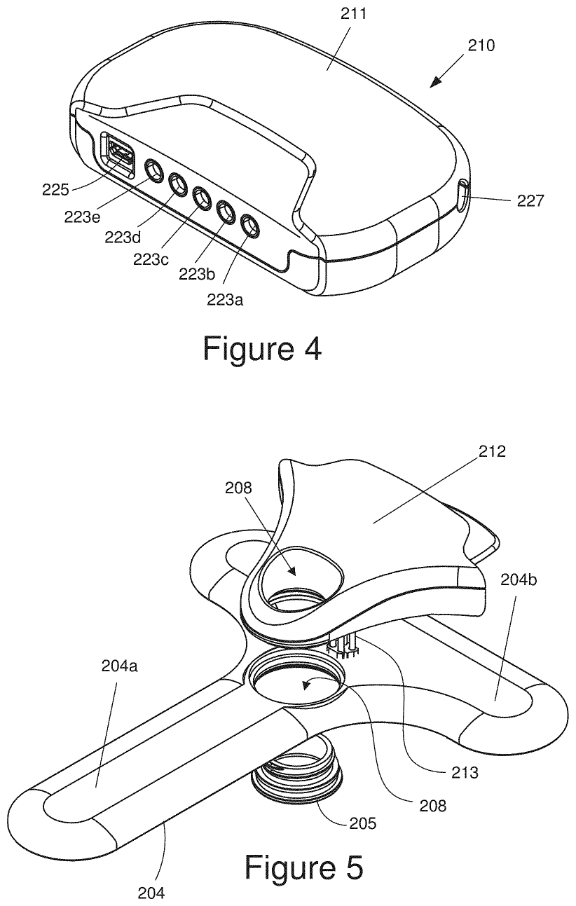

As will be described in more detail below, apparatus 10 includes a battery (for example, located in housing 211) for powering the components of the apparatus 10. For example, the battery may be a lithium-ion rechargeable battery. The battery may be charged via a USB port such as micro-USB port 225, or a DC input port 227 (FIG. 4). The level of charge in the battery may be displayed as an indicator to the user, for example by LED lights located in the housing 211, and/or on a display of a mobile device to which the apparatus 10 is paired.

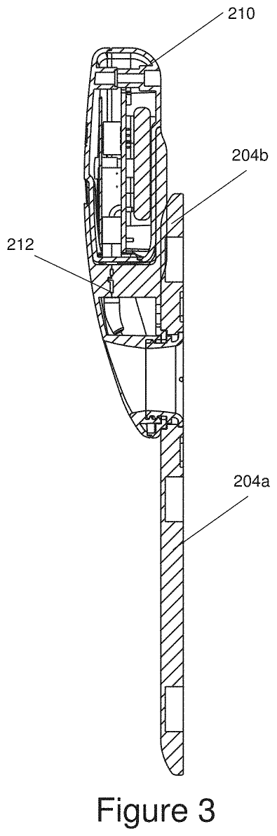

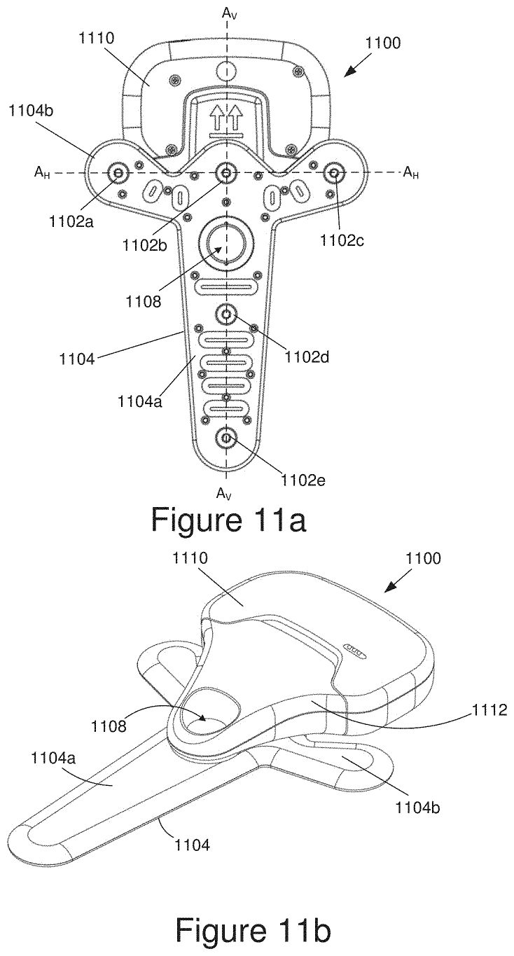

Turning to FIGS. 11a and 11b, an alternative embodiment of an apparatus 1100 for acquiring signals from a pregnant subject is shown. In FIGS. 11a and 11b, the reference numerals are incremented by 900 relative to FIGS. 1-7, 9 and 10, and have the same functions as their counterparts in those figures, with the exception of the flexible portion 1104, which is shaped somewhat differently. Both the first section 1104b and second section 1104a are still generally linear, so as to enable the signal acquisition points 1102a, 1102b, 1102c, 1102d, 1102e to form a T-shape as before. However, it can be seen that the first section 1104b has an undulating upper contour, while the second section 1104a is tapered along the axis A_{V} in a direction away from the intersection between axes A_{H} and A_{V}. The undulating upper contour of the first section 1104b enables further reduction of the footprint, and thus skin contact, of the apparatus 1100, and also enables greater flexibility of the first section 1104b and thus greater ease of conforming the flexible portion 1104 to the surface of the abdomen during use. The tapering of the second section 1104a also provides a smaller device footprint on the abdominal area.

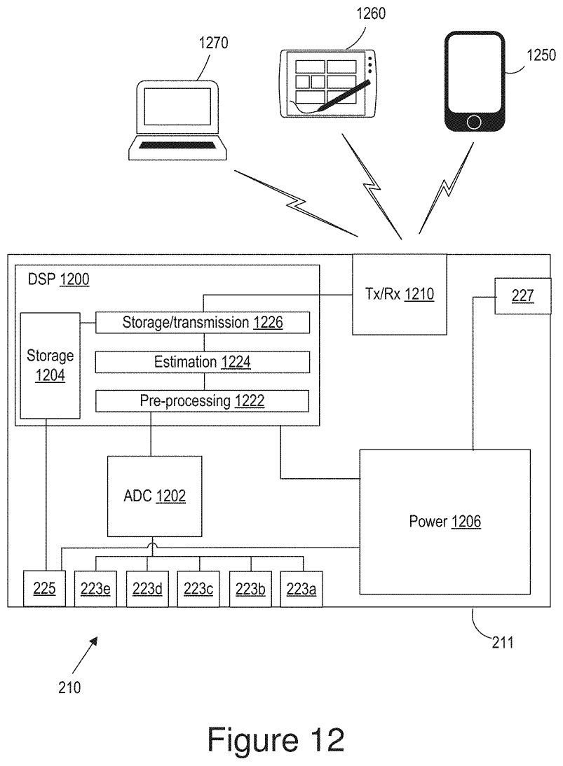

Referring now to FIG. 12, an exemplary block architecture of a signal processing module 210 of the apparatus 10 is shown. A similar block architecture may be employed in the apparatus 1100 of FIGS. 11a and 11b.

As discussed above, signal processing module 210 has a housing 211 that contains electrical components for receiving and processing signals generated at the signal acquisition points 202a, 202b, 202c, 202d, 202e by electrical activity at the skin of the subject. Raw signals from the signal acquisition points may be received at respective ports 223a, 223b, 223c, 223d, 223e. The raw signals may be fed to an ADC module 1202 which may filter and/or amplify the raw signals, and digitise them to pass to a digital signal processor (DSP 1200).

Signal processing module 210 may have a power source 1206, such as a Lithium-ion battery as mentioned earlier. Power source 1206 may be charged via DC input 227, or even by micro-USB port 225, for example.

The DSP 1200 may comprise various components including a pre-processing unit 1222 that applies one or more pre-processing algorithms, such as noise reduction, to the digitised signals; an estimation unit 1224 that derives parameters from the pre-processed digitised signals, such as one or more maternal health parameters or one or more fetal health parameters, such as fetal heartbeat; and a storage and transmission unit 1226 that stores raw and/or processed data at DSP 1200, and/or transmits raw and/or processed data, via a transmitter or transceiver 1210, to an external system, such as a smartphone 1250, a tablet 1260, or a laptop computer 1270.

For example, smartphone 1250 may execute an application that enables a user of the apparatus 10 to pair to the signal processing module 210 to receive processed data therefrom, such as the aforementioned health parameters, and to display the data in real-time. To this end, transceiver 1210 may be a Bluetooth™ transceiver that enables such pairing and data transfer to occur, under the control of storage/transmission unit 1226 of DSP 1200, for example.

In some embodiments, DSP 1200 may have, or be in communication with, on-board storage 1204, that can store raw and/or processed signals. On-board storage 1204 may be connected to micro-USB port 225 such that data can also be retrieved by a wired connection to an external computing device.

Process for Selection of the Contact Positions

Optimisation of the electrode (signal acquisition point) pattern of embodiments will now be described with reference to FIGS. 8a and 8b.

Using unipolar ECG leads where the reference is sufficiently far away from the measuring electrode may improve AFECG SNR. A unipolar lead implies that the collected signal is single-ended and is a potential difference between a reference (indifferent) electrode and a measuring electrode. However, unipolar leads may be affected by larger sensitivity to background noise such as power line interference and physiological artifacts such as electromyogram (EMG) signals and maternal ECG. Therefore, it may be beneficial to identify the appropriate distance between the reference and measuring electrodes.

During pregnancy, the uterus size typically increases from about 10 cm to about 60 cm. The average uterus size is determined by the gestational age (GA), which is determined in weeks, where GA of 37 weeks and above is at term. Additionally, the fetus position is variable as the fetus is mobile and changes position regularly within the womb. Furthermore, mothers may have different body mass index (BMI) and abdomen size. Therefore, it is important to design the shape of apparatus 10 or 1100 and the placement of the signal acquisition points 202 to be able to acquire accurate signals despite various abdomen sizes (which may depend on the size of the uterus, mobility of the fetus, and a wide range of BMI values).

To minimize patient discomfort, the total number of electrodes is preferably kept at a minimum. Additionally, this may result in a more compact device which is simpler to use. An ideal electrode position pattern should consider all three aspects: a large fetal ECG SNR, assurance of effective signal detection for various fetal positions, GA, and BMI, and minimal contact with the abdomen for the patient's comfort.

Uterine activity (UA), or contractions, can be obtained by measuring the uterine EMG signal, also known as electrohysterogram (EHG). The EHG can be measured using a unipolar lead where the reference and the measuring electrodes are close to each other. In this case, it is important that the measuring electrode is placed directly on the uterine muscle, or as close to as possible, over the diastasis of the rectus abdominis, with minimal interference from other abdominal muscles to avoid strong physiological artifacts.

An initial contact pattern was selected (FIG. 8a). The initial pattern is designed to use a single reference electrode 402ref at the bottom of the abdomen, for example 2 to 5 cm over the symphis pubis. The pattern of the measuring electrodes 402a, 402b, 402c, 402d, 402e, 402f was determined following an initial clinical study where electrodes were systematically positioned around the navel at various distances and angles. The position of the uterine contraction electrode 402ua was determined following the criteria described above.

Preferably, off-the-shelf wet gel electrodes developed for sensitive skin, such as 3M Red Dot electrodes 2238 or 2239, are used. For example, the wet gel electrodes may be circular with a 3 cm diameter.

A clinical study was performed using the initial pattern as shown in FIG. 8a, and involved n=30 patients with BMI ranging between 22 and 61 and GA between 25 to 41 weeks. For each measurement, the fetus position and presentation were measured using ultrasound and were statistically representative of reported world distribution.

A Signal Quality Index (SQI) was used to evaluate each electrode position on the initial pattern, similar to the SQI method used by Behar et al. (Behar et al., Physiol. Meas., 37 (2016) R1), the content of which is hereby incorporated herein by reference. The SQI provides a relative measure of signal quality within a single dataset and relies on dataset-dependent parameters. For each dataset, the electrode positions were ranked from 1 to 6, corresponding to the highest to lowest calculated SQI values, respectively. This provided a robust classification method of electrode positions A to F for statistical comparison between the datasets (Table 1).

TABLE 1
Statistical ranking of the electrode position according
to their SQI values across n = 30 datasets.

| Electrode Position | A | B | C | D | E | F |
|---|---|---|---|---|---|---|
| SQI rank | 5 | 6 | 1 | 4 | 3 | 2 |

Positions C, D, E, and F were identified with the highest ranks. Ranking of position F decreases with earlier GA, and electrode placements between position D and F are generally acceptable for AFECG acquisition. Therefore, the best electrode placement for AFECG and UA acquisition, that is able to cover the widest range of GAs and that minimises the device footprint (and thus the degree of discomfort to the patient) resembles the pattern of FIG. 8b.

The 3 fetal ECG electrodes 202a, 202b, 202c selected were the 3 side-by-side located just above the navel 18. This configuration satisfies the criteria of high SNR, various fetal position, GA, and BMI, and provides a compact and usable pattern for an improved patient comfort.

The selected shape for embodiments of the present invention resembles the one of a “T”, where the navel 18 is centrally located and provides a reference point for positioning and placement.

Electrode Contact Signal Quality Index (ECS)

Obtaining high quality abdominal electrophysiological signals is important to ensure that the apparatus collects an optimal bio-signal. If the electrodes are not applied and/or stuck properly to the subject's abdomen, it results in poor signal quality, decreasing the accuracy and performance of the bio-signal processing. Thus, it is important to ensure a satisfactory electrical contact with the abdominal skin of the subject.

To minimise the risk of the electrodes not being properly stuck to the subject's abdomen during use, an Electrode Contact Signal Quality Index (ECS) may be determined. The ECS helps to ensure that the disposable electrodes are adhered properly to the subject's abdomen by confirming a sufficient electrode contact with the skin to acquire optimal bio-signals. This feature serves as a risk mitigation action by notifying the subject to place, stick and use the electrodes correctly.

In certain embodiments, signal processing module 210, or an application executing on mobile device 1250 (for example) to which the signal processing module 210 is paired, may perform an electrode contact check for all 5 electrode contact points prior to the commencement of the monitoring session.

The signal transmitted from the apparatus 10 can be evaluated by assessing the power of the frequency range associated with a visible maternal QRS or with the frequency range of the electrode artifact.

For example, the raw ECG data acquired by signal processing module 210 can be analysed to determine a signal quality index (SQI), and a binary value output for each of the 5 channels to indicate if electrode contact to the user's abdomen is not detected. The detection result may be shown visually to the user on the mobile application.

The electrodes may be detected one by one, and if one or multiple electrodes are not stuck properly according to their respective SQI values, the mobile application may notify the subject to re-place the electrode(s).

An ECS algorithm may scan each electrode and calculate a frequency-based signal quality index (SQI). This is conceptually similar to signal-to-noise ratio (SNR). The ECS algorithm treats the SQI value as indicating “clean” data when it is higher than a certain value. As noise and SQI are inversely proportional, this value can be chosen to be the lowest value possible for thresholding the highest level of noise the algorithm server can process without compromising useful data.

$$S Q I = \frac{{Σ}_{n = 4}^{a} {X [ n ]}^{2}}{{Σ}_{n = a}^{b} {X [ n ]}^{2}} , where a = [ 50 \to 70 ] and b = [ 100 \to 150 ] ,$$

Where n is the Discrete Fourier Transform (DFT) index and X[n]² are the power spectrum values associated with the DFT values.

The SQI formula above is based on F. Andreotti, F. GraBer, H. Malberg and S. Zaunseder, “Non-invasive Fetal ECG Signal Quality Assessment for Multichannel Heart Rate Estimation,” in IEEE Transactions on Biomedical Engineering, vol. 64, no. 12, pp. 2793-2802, December 2017, followed by further analysis of frequency components through wavelet decomposition of 7 layers. The SQI used herein is a ratio of a sum of low frequency components (e.g., a up to 61 Hz) to a sum of high frequency components (e.g., b up to 125 Hz, as determined by the Nyquist-Shannon sampling theorem for a sampling rate of 250 Hz). In the numerator, the sum begins at n=4 to ensure that baseline and baseline wander are not included. In the denominator, the sum may begin at, e.g., a=61, since the range 61 Hz to 125 Hz typically contains high frequency noise such as electromyography (EMG), and artefacts resembling white Gaussian noise that would have a signal power outside the ECG range. There should be no ECG components within this range and it would contain irrelevant artefacts.

Hence, a ratio of these sums should be able to discriminate between signals with high frequency noise and clean signals (signals with minimal power outside the ECG band).

The higher the SQI value, the higher the quality of the signal. A suitable threshold for alerting the user to a low-quality signal may be SQI<=x, where x could range between 0<=x<=1. For example, a threshold value of x=1 could be used (i.e., SQI less than 1 would result in an alert notification to the user).

If the ECS check has passed, the monitoring session (for example, controlled by the application executing on smartphone 1250) may proceed immediately. The overall check will only pass if all individual electrodes pass the signal check.

If the ECS check has failed, the user may be notified via the mobile application, and the mobile application may indicate the necessary steps for repositioning and/or replacement of the electrodes. The specific electrodes which require attention may be highlighted. After taking the necessary steps, the user can proceed to re-initiate an ECS check.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that the prior art forms part of the common general knowledge.

1. An apparatus for acquiring signals from a pregnant subject, the apparatus including a flexible portion that is conformable to an abdomen of the subject, the flexible portion including a plurality of signal acquisition points, each signal acquisition point comprising a contact adapted to electrically couple to a respective electrode and signal acquisition circuitry, wherein the flexible portion includes:
a first generally linear section that is generally aligned along a first axis; and
a second generally linear section extending from the first linear section, the second linear section being generally aligned along a second axis that is perpendicular to the first axis;
wherein a first group of the signal acquisition points is located along the first axis and a second group of the signal acquisition points is located along the second axis.

2. The apparatus of claim 1, having a channel defined therethrough for aligning the apparatus with a navel of the subject.

3. The apparatus of claim 2, wherein the channel is positioned along the second axis.

4. The apparatus of claim 2, wherein at least one of the second group of signal acquisition points is located on one side of the channel, and at least one of the second group of signal acquisition points is located on the other side of the channel.

5. The apparatus of claim 2, wherein the channel defines a longitudinal axis that is mutually orthogonal to the first axis and the second axis.

6. The apparatus of claim 2, wherein the channel is shaped to allow a user to align the channel to the navel of the subject by visual and/or tactile means.

7. The apparatus of any one of claim 2, wherein the channel has a chamfered entry portion to facilitate gripping by a user.

8. The apparatus of claim 4, wherein one of the signal acquisition points on the other side of the channel is a uterine activity signal acquisition point, optionally wherein the uterine activity signal acquisition point is spaced about 8 cm from the intersection between the first axis and the second axis.

9. The apparatus of claim 4, wherein one of the signal acquisition points on the other side of the channel is a reference signal acquisition point located along the second axis to acquire reference signals, optionally wherein the reference signal acquisition point is located about 18 cm from the intersection between the first axis and the second axis.

10. The apparatus of claim 1, including a cable hub attached to the flexible portion for feeding signals from the signal acquisition points to the signal acquisition circuitry and/or to signal processing circuitry.

11. The apparatus of claim 10, wherein the cable hub is formed from a rigid material.

12. The apparatus of claim 10, wherein the channel extends through the cable hub and the flexible portion.

13. The apparatus of claim 10, including a signal processing module attached to or integral with the cable hub, the signal processing module comprising signal processing circuitry.

14. The apparatus of claim 1, comprising circuitry that is configured to determine a signal quality index (SQI) for each of a plurality of signals acquired at the signal acquisition points; and to generate an alert for any signal acquisition points for which the SQI falls below a predetermined threshold.

15. A system for acquiring signals from a pregnant subject, comprising:
a plurality of electrodes disposed on a flexible portion that is conformable to an abdomen of the subject, the plurality of electrodes being arranged in a T-shaped pattern in which a first group of electrodes is aligned along a first axis and a second group of electrodes is aligned along a second axis that is perpendicular to the first axis;
a rigid body that is connected to the flexible portion for alignment of the flexible portion on the abdomen of the subject; and
signal acquisition circuitry electrically coupled to said electrodes for acquiring electrocardiographic and/or electromyographic signals from the subject.

16. The system of claim 15, wherein the rigid body and flexible portion have a channel defined therethrough for aligning the apparatus with a navel of the subject.

17. The system of claim 16, wherein the channel has a chamfered entry portion to facilitate gripping by a user.

18. The system of claim 16, wherein at least one of the second group of electrodes is located on one side of the channel, and at least one of the second group of electrodes is located on the other side of the channel, such that in use, at least one electrode lies above the navel and at least one electrode lies below the navel.

19. The system of claim 18, wherein one of the signal acquisition points on the other side of the channel is a uterine activity signal acquisition point, optionally wherein the uterine activity signal acquisition point is spaced about 8 cm from an intersection between the first axis and the second axis.

20. The system of claim 18, wherein one of the signal acquisition points on the other side of the channel is a reference signal acquisition point located along the second axis to acquire reference signals, optionally wherein the reference signal acquisition point is spaced about 18 cm from an intersection between the first axis and the second axis.
